(19) United States
(12) Patent Application Publication
(10) Pub. No.: US 2001/0031954 A1
(43) Pub. Date: Oct. 18, 2001

(54) Absorbent articles with enhanced graphic impact

(76) Inventors: Joy Francine Jordan, Marietta, GA (US); Julie Ann George, Appleton, WI (US); Raymond Jeffrey May, Norcross, GA (US); Jennifer Leigh Skabroud Misek, Little Chute, WI (US); Carl Gerard Rippl, Appleton, WI (US)

Correspondence Address: Thomas M. Gage, Kimberly-Clark Worldwide, Inc., Patent Department, 401 North Lake Street, Neenah, WI, 54956, US

(21) Appl. No.: 09/736,527

(22) Filed: Dec. 13, 2000

Related U.S. Application Data
(60) Provisional application No. 60/174,462, filed on Jan. 5, 2000.

(51) Int. Cl. A61F 13/15; A61F 13/20

(52) U.S. Cl. 604/385.01; 604/361; 604/364; 604/365; 604/372

(57) ABSTRACT

An absorbent article includes an outer cover graphic that appears brighter and more noticeable than present outer cover graphics. The outer cover includes a liquid impermeable inner layer and a fibrous outer layer which layers jointly define a graphic region where the inner and outer layers are intimately bonded together. The fibrous outer layer has a Light Transmittance Value in the graphic region of about 80 percent or higher, and an outer cover graphic is disposed on the inner layer in a location corresponding to the graphic region.

### BACKGROUND OF THE INVENTION

The present invention relates to absorbent articles which are adapted to contain body exudates. More particularly, the invention pertains to disposable absorbent articles with improved outer cover graphics.

With increasing frequency disposable absorbent articles are incorporating graphics as a component of the product. For the most part, these graphics have been applied to the outer cover of the product so that the graphics are visible while the product is being worn. The graphics have been printed directly on a component of the outer cover or have been printed on a separate layer, such as a tissue layer, which is disposed on the outer cover.

There are many reasons to incorporate graphics in disposable absorbent articles. Most simply, the graphics can improve the appearance and appeal of the product, to both the wearer and the purchaser. Graphics can also impact the manner in which a disposable absorbent article is used. By way of illustration, graphics on disposable diapers can be used by caregivers to amuse and/or educate the child during diapering. Similarly, graphics on disposable training pants can provide educational and motivational mechanisms to facilitate the toilet training process. Graphics on training pants can also serve to increase the child's interest in the product and thereby increase the child's interest in the toilet training process.

Graphics can also be used to indicate when a urine insult has occurred, for example by changing the appearance of the graphic upon exposure to liquid. Such active graphics can be useful in adult incontinence products, diapers and training pants. Disposable absorbent articles can also incorporate graphics to indicate the front or back of the product, instruct the user as to use of the absorbent article, or the like.

To date, various product and process constraints have limited the visual quality of graphics used on disposable absorbent articles. In particular, it is generally required that absorbent articles include a relatively quiet and cloth-like outer cover. There is also a need that outer covers for disposable absorbent articles be capable of high speed manufacture. These requirements have negatively impacted the visual quality of graphics in disposable absorbent articles. Thus, what is lacking and needed in the art is a disposable absorbent article with enhanced graphic impact.

### SUMMARY OF THE INVENTION

In response to the above-referenced unfulfilled need in the art, a new disposable absorbent article has been discovered. The absorbent article includes an outer cover graphic that appears brighter and more noticeable than presently exists with known outer cover graphics.

In one embodiment, the present invention pertains to an absorbent article including an absorbent assembly having a major surface and an outer cover positioned against the major surface of the absorbent assembly. The outer cover comprises a liquid impermeable inner layer and a fibrous outer layer. The inner and outer layers jointly define at least one graphic region where inner and outer layers are intimately bonded together. In the at least one graphic region, the fibrous outer layer has a Light Transmittance Value of about 80 percent or higher. An outer cover graphic of the absorbent article is disposed on the inner layer in a location that corresponds to the at least one graphic region.

With this feature of the invention, the outer cover graphic is highly noticeable to the user and others viewing the absorbent article due to the positioning of the graphic in the graphic region and the light transmittance characteristics of the outer layer.

The inner and outer layers of the outer cover can be bonded together by a variety of means known in the art, including thermal bonds, adhesive bonds, ultrasonic lamination, or the like. Intimate bonding of the inner and outer layers in the graphic regions reduces light diffraction and thus improves the brightness and overall visibility of the outer cover graphic. As used herein, the term “intimate bonding” refers to physical contact between two layers such that they resist separation with no readily visually identifiable areas of separation. In one particular embodiment, the inner and outer layers are adhesively laminated together in the graphic regions using a meltblowing process to form an overlapping network of adhesive filaments. Adhesive bonding can also be accomplished using adhesive slot coating, high frequency oscillation patterns, for example in swirl or spray patterns, and other fine denier and/or high coverage application techniques. Suitable laminate adhesives, which can be applied continuously or intermittently, can be obtained from Findley Adhesives, Inc., of Wauwatosa, Wis. U.S.A. or from National Starch and Chemical Company, Bridgewater, N.J. U.S.A.

The outer layer of the outer cover is selected to provide a markedly higher degree of light transmittance than conventional outer cover nonwoven fabrics. The outer layer of the present invention suitably has a Light Transmittance Value, measured according to the procedure described hereinafter, of about 80 percent or higher, such as from about 80 to about 95 percent, particularly about 82 or higher more, such as from about 82 to about 95 percent, and more particularly about 84 percent or higher, such as from about 84 to about 88 percent.

The outer layer can be made in a variety of forms using different process. For example, the outer layer may be formed as a carded web, a bonded carded web, a spunbond web, a needled fabric, a woven fabric, or the like to provide a generally cloth-like texture to the wearer. The outer layer can comprise from about 99.5 to 100 weight percent polymer resin and a limited amount of other additives which assist with processing and do not significantly alter the Light Transmittance Value of the material. The other additives such as titanium dioxide can represent about 0.5 weight percent or less, particularly about 0.3 weight percent or less, of the outer layer. In one particular embodiment, the outer layer comprises a spunbond web formed of about 99.5 to 100 weight percent polypropylene resin and about 0.5 weight percent or less other additives. The outer layer is desirably a lightweight material having a basis weight of about 13 to about 34 grams per square meter (about 0.4-1 osy), and particularly from about 17 to about 20 grams per square meter (about 0.5-0.6 osy).

The inner layer is desirably manufactured from a thin plastic film, for example, films formed of polyethylene, polypropylene, catalloy, bi-component, any polymer based extruded film, or the like, although other flexible liquid impermeable materials can also be used. The inner layer prevents waste material from wetting articles, such as bedsheets and clothing, as well as the wearer and caregiver. A suitable liquid impermeable film for use as liquid impermeable inner layer is a 0.025 millimeter (1.0 mil) polyethylene film commercially available from Pliant Corporation of South Plainfield, N.J. U.S.A. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn.

In another embodiment, the present invention pertains to an absorbent article including an absorbent assembly, a liquid permeable topsheet disposed on the absorbent assembly, and an outer cover bonded to the topsheet and sandwiching the absorbent assembly between the topsheet and the outer cover. The outer cover comprises a liquid impermeable inner layer and a fibrous outer layer. The inner and outer layers jointly define at least one graphic region where inner and outer layers are intimately bonded together. The fibrous outer layer has a Light Transmittance Value in the at least one graphic region of about 82 percent or higher. The absorbent article also includes an active graphic disposed on the inner layer in liquid communication with the absorbent assembly and in a location that corresponds to the at least one graphic region.

In particular embodiments, the graphics of the absorbent article include active graphics that are constructed to “appear” or “disappear” from view. The term “active graphic” as used herein refers to an appearing graphic, a fading graphic, or a combination of appearing and fading graphics. The term “appearing graphic” is used herein to refer to a graphic that becomes visible or becomes significantly more visible when exposed to urine, or that becomes visible or becomes significantly more visible with the passage of time when exposed to the environment but not exposed to urine. Conversely, the term “fading graphic” is used herein to refer to a graphic that becomes invisible or significantly less visible when exposed to urine, or that becomes invisible or significantly less visible with the passage of time when exposed to the environment but not exposed to urine. Active graphics can be useful in training pants for example to inform the caregiver and/or child when an accident has occurred.

In particular embodiments, the active graphic can comprise a fading graphic which is formed from an ink that is soluble in aqueous solutions such as urine. The ink is positioned in the absorbent article so that it becomes wet and dissolves when the product is insulted with liquid. Once dissolved, the ink washes away from the outer cover and is obscured by the outer cover. As a result, the active graphic seems to disappear from view. Suitable urine-soluble inks are available from a variety of commercial vendors, such as Sun Chemical Corp. of Philadelphia, Pennsylvania U.S.A. under the trade designation AQUA DESTRUCT. Particular urine-soluble compositions are disclosed in U.S. Pat. No. 4,022,211 issued May 10, 1977 to Timmons et al., which is incorporated herein by reference. The ink color can be selected to provide a pleasing appearance and graphic impact, including fading rapidly upon contact with liquid. To facilitate rapid fading, the fading graphics can comprise line drawings having a line width of from about 1 to about 2 millimeters.

The active graphic can also comprise a fading or an appearing graphic which is formed from a composition such as an ink or adhesive that changes color when exposed to an aqueous solution such as urine. A color change composition can be adapted to blend in with a background or surrounding color, either before or after exposure to the aqueous solution. Suitable compositions of this color-change type are available from a variety of commercial vendors, such as a pH-change/color-change hot melt adhesive available from Findley Adhesives, Inc. of Wauwatosa, Wis. U.S.A. Alternatively, the active graphic can comprise pH sensitive inks, fugitive inks, colored absorbent particles, hydratable salts, moisture sensitive films, enzymes, heat sensitive inks and dyes, or the like.

Fading graphics can simply disappear from view, relative to the exterior surface of the outer cover. For example, the graphics can also comprise permanent graphics so that the fading object graphics can be made to disappear into a permanent background graphic. One representative example is an object graphic such as yellow fish disappearing or fading into a background graphic such as blue water or green weeds.

The active graphic can also be configured to appear over time due to exposure to the environment. In particular, the active graphic can be responsive to time intervals, temperature levels, oxygen levels, or the like, and combinations thereof. Various visual indicators that appear over time in response to particular conditions are disclosed in U.S. Pat. No. 5,058,088 issued Oct. 15, 1991 to Haas et al.; U.S. Pat. No. 5,053,339 issued Oct. 1, 1991 to Patel; U.S. Pat. No. 5,045,283 issued Sep. 3, 1991 to Patel; U.S. Pat. No. 4,987,849 issued Jan. 29, 1991 to Sherman; U.S. Pat. No. 4,903,254 issued Feb. 20, 1990 to Haas; U.S. Pat. No. 4,812,053 issued Mar. 14, 1989 to Bhattacharjee; and U.S. Pat. No. 4,292,916 issued Oct. 6, 1981 to Bradley et al.; all of which are incorporated herein by reference. An active graphic that appears over time may be applied to the product when use is initiated, or formed as an integral component of the product.

In contrast to active graphics, the term “permanent graphic” is used herein to refer to a graphic that does not substantially change its degree of visibility when the absorbent article is insulted with urine and when the absorbent article is exposed to the environment, in simulated use conditions. The change in visibility of a graphic or a portion of a graphic can be determined based on a person's observation of the graphic before and after the article containing the graphic is exposed to liquid.

Various configurations of character and object graphics that are specifically adapted for training pants to enhance the toilet training process are disclosed in U.S. patent application Ser. No. 09/333,222 filed on Jun. 15, 1999 by C. P. Olson et al. and titled “Absorbent Articles Having Wetness Indicating Graphics Providing An Interactive Training Aid;” U.S. patent application Ser. No. 09/333,223 filed on Jun. 15, 1999 by M. T. Cammarota et al. and titled “Absorbent Articles Having Wetness Indicating Graphics Incorporating A Training Zone;” and U.S. patent application Ser. No. 09/444,081 filed on Nov. 22, 1999 by S. A. Weber et al. and titled “Absorbent Articles Including Hidden Graphics;” which are incorporated herein by reference. The permanent and active graphics are believed to make children more interested in the toilet training process and therefore lead to enhanced results. The changed condition of the active graphic presents a tool for the caregiver to interact with the child and explain why the graphic changed. This is particularly useful at the stage of toilet training where the child is being taught to be aware of going potty and the need to use the bathroom.

Outer cover graphics are directly visible on the exterior surface of the absorbent article and have been extremely appealing to children. Moreover, parents and caregivers can use outer cover graphics as educational and motivational tools to advance the toilet training process. Thus, the permanent and active graphics are suitably disposed on the outer cover. The term “disposed on” and variations thereof are intended to mean that one element can be integral with another element, or that one element can be a separate structure bonded to or placed with or placed near another element. Thus, the graphics can be formed or applied directly or indirectly to a surface of the outer cover, formed or applied between layers of a multiple layer outer cover, formed or applied to a substrate that is placed with or near the outer cover, formed or applied within a layer of the outer cover or another substrate, or other variations or combinations thereof. In particular embodiments, the graphics can be printed, sprayed, or otherwise applied directly on a layer of the outer cover. In other embodiments, the graphics can be applied to a layer placed with or near the outer cover, such as a substrate associated with the absorbent assembly, including but not limited to tissue layers, liquid handling layers, absorbent layers, or the like.

The graphics for use with the present invention can be located on or against either surface of the inner layer of the outer cover, on or against the interior surface of the outer layer, or between the inner and outer layers, provided the graphics remain visible from the exterior of the product.

The permanent and active graphics can be formed on or applied to the outer cover or another substrate bonded to or placed with or placed near the outer cover by any suitable technique. The graphics are desirably registered with other components of the absorbent article during manufacture such that the graphics are positioned in the desired regions of the product.

For active graphics that are triggered by contact with urine, the active graphic can be in liquid communication with the absorbent assembly of the product. As used herein, the term “liquid communication” means that liquid such as urine is able to travel from one layer or element to another layer or element. The absorbent assembly can but need not include a slot or densified region, incorporate a liquid distribution layer, or the like, to channel or direct liquid to the location near the outer cover where the active graphics are located.

As noted previously, liquid soluble inks can be used to form the active graphics. It is theorized that migration of the dissolved inks away from the outer cover and into the absorbent assembly can improve the fading or disappearing quality of the active graphics. To enhance this effect, the outer cover can be attached to the absorbent assembly in a windowpane design, whereby the active graphic region of the outer cover is not bonded to the absorbent assembly and the regions of the outer cover surrounding the active graphic region are bonded to the absorbent assembly. One suitable method and apparatus for adhesively bonding the outer cover to the absorbent assembly in a windowpane design is disclosed in U.S. Pat. No. 5,683,752 issued Nov. 4, 1997 to Popp et al., which is incorporated herein by reference.

Absorbent articles suitable for use with the present invention include diapers, training pants, incontinence products, diaper pants, disposable underwear, or the like. Suitable training pants and diaper pants can have seamed side portions or refastenable side portions. The present invention is particularly suited for use with training pants or diaper pants to aid in toilet training. Particular training pants suitable for use with the present invention are disclosed in U.S. Pat. No. 4,940,464 issued Jul. 10, 1990 to Van Gompel et al.; and U.S. patent application Ser. No. 09/444,083, filed on Nov. 22, 1999 by A. L. Fletcher et al. and titled “Absorbent Articles With Refastenable Side Seams;” which are incorporated herein by reference. These references describe various materials and methods for constructing training pants. The training pant can also be constructed using the methods and apparatus disclosed in U.S. Pat. No. 5,766,389 issued Jun. 16, 1998 to Brandon et al., which is also incorporated herein by reference.

The above-mentioned and other features and advantages of the present invention and the manner of attaining them will become more apparent, and the invention itself will be better understood by reference to the drawings and the following description of the drawings.

### Definitions

Within the context of this specification, each term or phrase below will include the following meaning or meanings.

“Bonded” refers to the joining, adhering, connecting, attaching, or the like, of two elements. Two elements will be considered to be bonded together when they are bonded directly to one another or indirectly to one another, such as when each is directly bonded to intermediate elements.

“Comprising” is inclusive or open-ended and does not exclude additional, unrecited elements or method steps.

“Connected” refers to the joining, adhering, bonding, attaching, or the like, of two elements. Two elements will be considered to be connected together when they are connected directly to one another or indirectly to one another, such as when each is directly connected to intermediate elements.

“Disposable” refers to articles which are designed to be discarded after a limited use rather than being laundered or otherwise restored for reuse.

“Disposed,” “disposed on” and variations thereof are intended to mean that one element can be integral with another element, or that one element can be a separate structure bonded to or placed with or placed near another element.

“Elastic,” “elasticized” and “elasticity” mean that property of a material or composite by virtue of which it tends to recover its original size and shape after removal of a force causing a deformation.

“Elastomeric” refers to a material or composite which can be elongated by at least 25 percent of its relaxed length and which will recover, upon release of the applied force, at least 10 percent of its elongation. It is generally preferred that the elastomeric material or composite be capable of being elongated by at least 100 percent, more preferably by at least 300 percent, of its relaxed length and recover, upon release of an applied force, at least 50 percent of its elongation.

“Fabrics” is used to refer to all of the woven, knitted and nonwoven fibrous webs.

“Flexible” refers to materials which are compliant and which will readily conform to the general shape and contours of the wearer's body.

“Force” includes a physical influence exerted by one body on another which produces acceleration of bodies that are free to move and deformation of bodies that are not free to move. Force is expressed in grams per unit area.

“Graphic” refers to any design, pattern, or the like that is or becomes visible on an absorbent article, and specifically includes text messages that consist of one or more alphanumeric symbols and pictorial images that consist of one or more pictures.

“Hydrophilic” describes fibers or the surfaces of fibers which are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90° are designated “wettable” or hydrophilic, while fibers having contact angles greater than 90° are designated “nonwettable” or hydrophobic.

“Integral” is used to refer to various portions of a single unitary element rather than separate structures bonded to or placed with or placed near one another.

“Inward” and “outward” refer to positions relative to the center of an absorbent article, and particularly transversely and/or longitudinally closer to or away from the longitudinal and transverse center of the absorbent article.

“Layer” when used in the singular can have the dual meaning of a single element or a plurality of elements.

“Liquid impermeable”, when used in describing a layer or multi-layer laminate, means that a liquid, such as urine, will not pass through the layer or laminate, under ordinary use conditions, in a direction generally perpendicular to the plane of the layer or laminate at the point of liquid contact. Liquid, or urine, may spread or be transported parallel to the plane of the liquid impermeable layer or laminate, but this is not considered to be within the meaning of “liquid impermeable” when used herein.

“Longitudinal” and “transverse” have their customary meaning, as indicated by the longitudinal and transverse axes depicted in FIG. 2. The longitudinal axis lies in the plane of the article and is generally parallel to a vertical plane that bisects a standing wearer into left and right body halves when the article is worn. The transverse axis lies in the plane of the article generally perpendicular to the longitudinal axis. The article as illustrated is longer in the longitudinal direction than in the transverse direction.

“Member” when used in the singular can have the dual meaning of a single element or a plurality of elements.

“Nonwoven web” means a web of material which is formed without the aid of a textile weaving or knitting process.

“Operatively joined,” with reference to the attachment of an elastic member to another element, means that the elastic member when attached to or connected to the element, or treated with heat or chemicals, by stretching, or the like, gives the element elastic properties; and with reference to the attachment of a non-elastic member to another element, means that the member and element can be attached in any suitable manner that permits or allows them to perform the intended or described function of the joinder. The joining, attaching, connecting or the like can be either directly, such as joining either member directly to an element, or can be indirectly by means of another member disposed between the first member and the first element.

“Outer cover graphic” refers to a graphic that is directly visible upon inspection of is the exterior surface of a garment, and for a refastenable garment is in reference to inspection of the exterior surface of the garment when the fastening system is engaged as it would be during use. The outer cover graphic may comprise a text message, a pictorial image, or a combination of the two.

“Permanently bonded” refers to the joining, adhering, connecting, attaching, or the like, of two elements of an absorbent garment such that the elements tend to be and remain bonded during normal use conditions of the absorbent garment.

“Pictorial image” means a graphic consisting of one or more pictures; the terms “text message” and “pictorial image” are mutually exclusive as used herein.

“Refastenable” refers to the property of two elements being capable of releasable attachment, separation, and subsequent releasable reattachment without substantial permanent deformation or rupture.

“Releasably attached,” “releasably engaged” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment.

“Rupture” means the breaking or tearing apart of a material; in tensile testing, the term refers to the total separation of a material into two parts either all at once or in stages, or the development of a hole in some materials.

“Stretch bonded” refers to an elastic member being bonded to another member while the elastic member is extended at least about 25 percent of its relaxed length. Desirably, the term “stretch bonded” refers to the situation wherein the elastic member is extended at least about 100 percent, and more desirably at least about 300 percent, of its relaxed length when it is bonded to the other member.

“Stretch bonded laminate” refers to a composite material having at least two layers in which one layer is a gatherable layer and the other layer is an elastic layer. The layers are joined together when the elastic layer is in an extended condition so that upon relaxing the layers, the gatherable layer is gathered.

“Surface” includes any layer, film, woven, nonwoven, laminate, composite, graphic, ink, or the like, whether pervious or impervious to air, gas, and/or liquids.

“Tension” includes a uniaxial force tending to cause the extension of a body or the balancing force within that body resisting the extension.

“Text message” means a graphic consisting of one or more alphanumeric symbols; the terms “text message” and “pictorial image” are mutually exclusive as used herein.

“Thermoplastic” describes a material that softens when exposed to heat and which substantially returns to a nonsoftened condition when cooled to room temperature.

These terms may be defined with additional language in the remaining portions of the specification.

### BRIEF DESCRIPTION OF THE DRAWINGS

The above-mentioned and other features of the present invention and the manner of attaining them will become more apparent, and the invention itself will be better understood by reference to the following description and the accompanying drawings, wherein similar features in different figures have been given the same reference numeral.

FIG. 1 illustrates a front perspective view of a training pant incorporating the principles of the present invention, showing both permanent and active graphics.

FIG. 2 illustrates a top plan, partially disassembled view of the outer surface of the training pant of FIG. 1, in a stretched and laid flat condition and with portions broken away for purposes of illustration.

FIG. 3 illustrates an enlarged and exploded section view of an absorbent chassis of the training pant of FIG. 1.

FIG. 4 illustrates a front perspective view similar to FIG. 1, although showing only the permanent graphics.

### DETAILED DESCRIPTION OF THE DRAWINGS

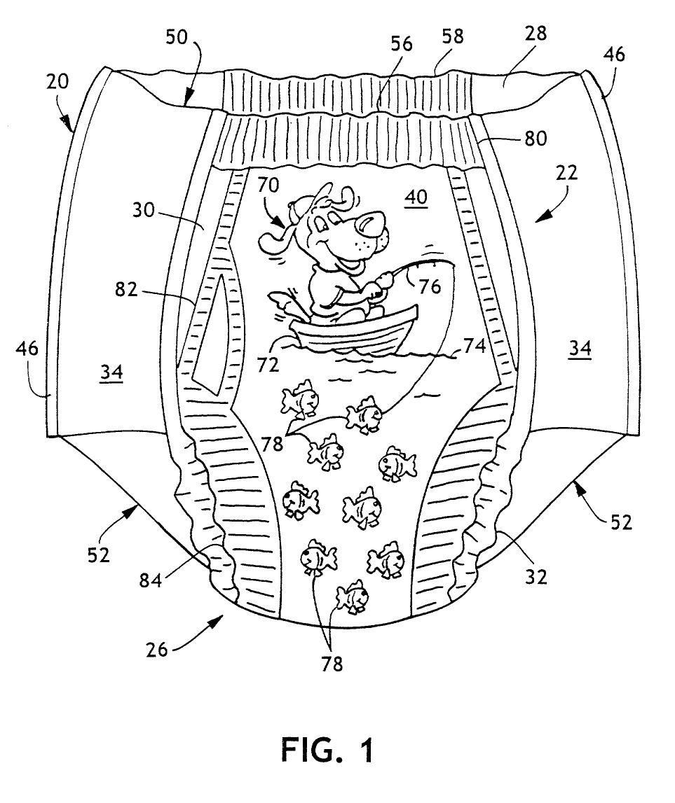

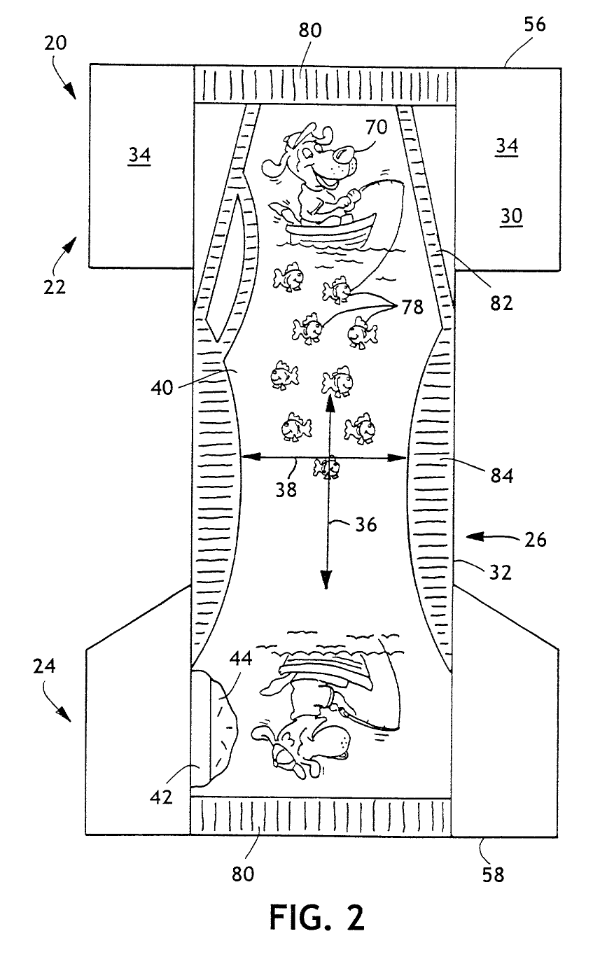

Although the principles of the present invention can be incorporated into a variety of absorbent articles, for ease of explanation the description hereafter will be in terms of a child's training pant 20, which is illustrated in a fully assembled condition in FIG. 1 and in a partially disassembled, stretched and laid flat condition in FIG. 2. The training pant 20 defines a first or front waist region 22, a second or back waist region 24, a crotch region 26 positioned between and interconnecting the front and back waist regions, an inner surface 28 (FIG. 1) which is configured to contact the wearer, and an outer surface 30 opposite the inner surface which is configured to contact the wearer's clothing. The illustrated training pant 20 comprises an absorbent chassis 32 and a plurality of transversely opposed side panels 34. The absorbent chassis 32 and side panels 34 can be integrally formed or comprise two or more separate elements, as shown.

With particular reference to FIG. 2, the training pant 20 defines a longitudinal centerline 36, a transverse centerline 38, a first or front longitudinal end edge 56, and a second or back longitudinal end edge 58. The first waist region 22 abuts the first longitudinal end edge 56, and the second waist region 24 abuts the second longitudinal end edge 58. The illustrated absorbent chassis 32 comprises an outer cover 40 and a bodyside liner 42 which is connected to the outer cover in a superposed relation. The absorbent chassis 32 also comprises an absorbent assembly 44 which is located between the outer cover and the bodyside liner, and can optionally include a pair of containment flaps (not shown).

With the training pant 20 in a fully assembled condition as illustrated in FIG. 1, the front and back waist regions 22 and 24 are joined together by side seams 46 to define a waist opening 50 and a pair of leg openings 52. The seams 46 can comprise non-refastenable seams, which may be formed by adhesive bonds, thermal bonds, ultrasonic bonds, or the like. Alternatively, the seams 46 can comprise refastenable fastening components, such as mating mechanical fasteners (not shown). Still alternatively, the training pant 20 can include both non-refastenable side seams 46 and refastenable fastening components (not shown).

The front waist region 22 comprises the portion of the training pant 20 which, when worn, is positioned on the front of the wearer while the back waist region 24 comprises the portion of the training pant which, when worn, is positioned on the back of the wearer. The crotch region 26 of the training pant 20 comprises the portion of the training pant which, when worn, is positioned between the legs of the wearer and covers the lower torso of the wearer. The side panels 34 comprise the portions of the training pant 20 which, when worn, are positioned on the side hip regions of the wearer. The longitudinal end edges 56 and 58 of the training pant 20 are configured to encircle the waist of the wearer when worn and provide the waist opening 50.

The absorbent chassis 32 is configured to contain and/or absorb any body exudates discharged from the wearer. For example, the absorbent chassis 32 desirably although not necessarily comprises the pair of containment flaps (not shown) which can be configured to provide a barrier to the transverse flow of body exudates. Suitable constructions and arrangements for the containment flaps are generally well known to those skilled in the art and are described in U.S. Pat. No. 4,704,116 issued Nov. 3, 1987 to Enloe, which is incorporated herein by reference.

To further enhance containment and/or absorption of body exudates, the training pant 20 can include a front waist elastic member, a rear waist elastic member, and leg elastic members (not shown), as are known to those skilled in the art. Waist elastic members and leg elastic members can be operatively joined to the outer cover 40 and/or bodyside liner 42 of the training pant 20. Elastic members for the containment flaps, waist elastics and leg elastics can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic constrictive forces are imparted to the substrate. In one particular embodiment, for example, the leg elastic members comprise a plurality of dry-spun coalesced multifilament spandex elastomeric threads sold under the trade name LYCRA® and available from E. I. Du Pont de Nemours and Company, Wilmington, Del. U.S.A.

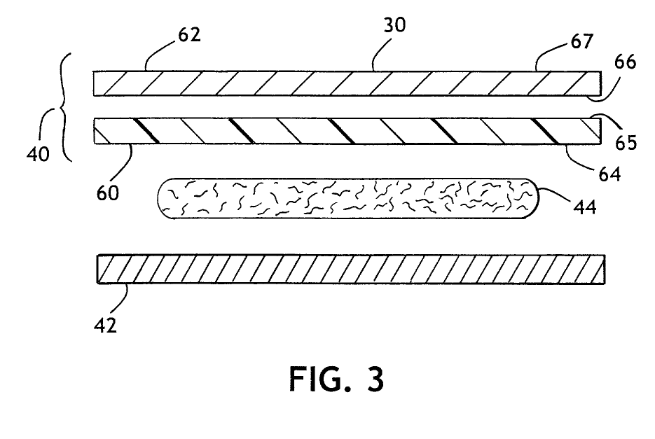

An enlarged and exploded section view of the absorbent chassis 32 is illustrated in FIG. 3. The absorbent assembly 44 is shown sandwiched between the outer cover 40 and the bodyside liner 42. The outer cover 40 comprises a multi-layered laminate structure including an inner layer 60 and an outer layer 62. The inner layer 60 has an interior surface 64 disposed toward the absorbent assembly 44 and an opposite exterior surface 65 disposed toward the outer layer 62. The outer layer 62 has an interior surface 66 disposed toward the inner layer 60 and an opposite exterior surface 67 that forms a portion of the outer surface 30 of the training pant 20. In particular embodiments the exterior surface 65 of the inner layer 60 is bonded to the interior surface 66 of the outer layer 62, for example, intimately bonded together at least in graphic regions where the outer cover graphics are located. In other particular embodiments, the bonded surface area between the inner and outer layers 60 and 62 can but need not necessarily be about 2 percent or more, such as about 15 percent or more, at least in the graphic regions. While the outer cover 40 is illustrated with two layers, it could alternatively comprise more than two individual layers (not shown).

As noted above, the outer layer 62 is selected to provide a markedly higher degree of light transmittance than conventional outer cover nonwoven fabrics. The outer layer of the present invention suitably has a Light Transmittance Value, measured according to the procedure described hereinafter, of about 80 percent or higher, such as from about 80 to about 95 percent, particularly about 82 or higher more, such as from about 82 to about 95 percent, and more particularly about 84 percent or higher, such as from about 84 to about 88 percent.

Various placements of the permanent and active graphics may be better understood with reference to FIG. 3. The permanent and active graphics can be disposed on the outer cover 40, which includes, in particular, on the interior surface 64 of the inner layer 60, on either or both facing surfaces 65 and 66 of the inner and outer layers 60 and 62, between the outer and inner layers 60 and 62, on the surface of the absorbent assembly 44 that faces the outer cover 40, or between the absorbent assembly and the outer cover. Graphics can be included on the outer surface 30 of the outer cover, although this would not take full advantage of the present invention.

In such embodiments and where the active graphics are triggered by contact with urine, it is desirable for the active graphics to be disposed on the interior surface 64 of the inner layer 60, on the surface of the absorbent assembly 44 that faces the outer cover, or between the absorbent assembly and the outer cover. The graphics can be incorporated within or on various layers of the absorbent assembly, such as tissue wrap sheets, liquid distribution or liquid handling layers, or the like. The permanent and active graphics need not be located in the same position or on the same substrate.

The illustrated training pant 20 (FIG. 1) includes registered outer cover graphics, including interactive wetness indicating graphics. More specifically, the training pant includes a permanent character graphic 70 in the form of a dog, permanent object graphics 72, 74 and 76 in the form of a boat, curved line segments denoting the surface of water, and a fishing pole, respectively, and a plurality of active object graphics 78 representing fish. The outer cover graphics also include a simulated elastic waistband 80, a simulated fly opening 82, and simulated elastic leg bands 84, all of which can be permanent graphics.

The active object graphics 78 can be disposed on the interior surface of the outer cover 40 and are visible from the exterior surface of the outer cover. The active object graphics 78 are in liquid communication with the absorbent assembly 44, meaning that liquid such as urine is capable of moving between the active object graphic and the absorbent assembly under ordinary use conditions.

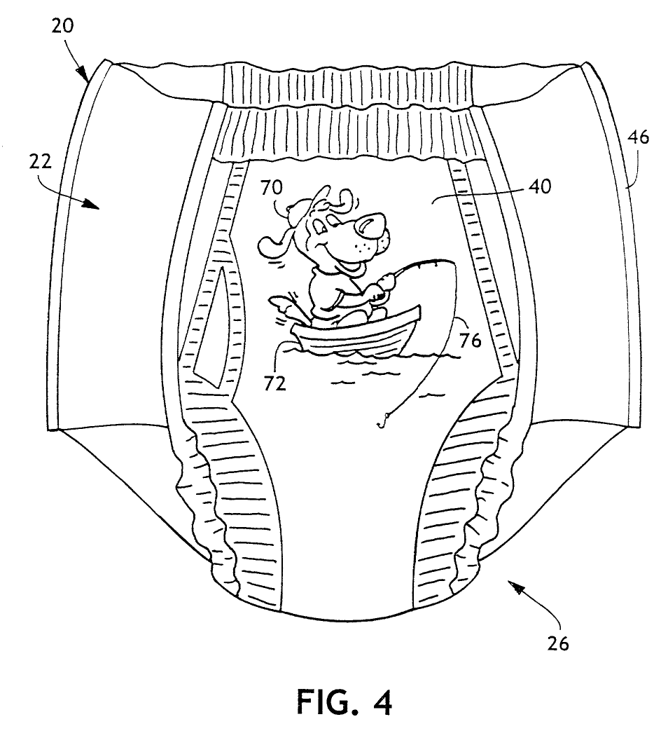

When the child wets the training pant 20, liquid is communicated to the active object graphics 78, whereupon the object graphics either dissolve, change color, appear, or the like. The effect is illustrated in FIG. 4, showing the situation where the active object graphics 78 disappear upon contact with urine. The front view of FIG. 4 shows that the active object graphics 78 are no longer present. Where appearing graphics are employed, the situation would work in reverse and the appearance of the pant would change from FIG. 4 to FIG. 1 upon liquid insult. Alternatively, the active object graphics 78 can comprise appearing graphics that are triggered upon use by exposure to the environment.

The liquid permeable bodyside liner 42 generally overlies the outer cover 40 and absorbent assembly 44, and can but need not have the same dimensions as the outer cover 40. The bodyside liner 42 is desirably compliant, soft feeling, and non-irritating to the child's skin. Further, the bodyside liner 42 can be less hydrophilic than the absorbent assembly 44, to present a relatively dry surface to the wearer and permit liquid to readily penetrate through its thickness.

The bodyside liner 42 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics can be used for the bodyside liner 42. For example, the bodyside liner can be composed of a meltblown or spunbonded web of polyolefin fibers. The bodyside liner can also be a bonded-carded web composed of natural and/or synthetic fibers. The bodyside liner can be composed of a substantially hydrophobic material, and the hydrophobic material may, optionally, be treated with a surfactant or otherwise processed to impart a desired level of wettability and hydrophilicity. For example, the material can be surface treated with about 0.45 weight percent of a surfactant mixture comprising Ahcovel N-62 from Hodgson Textile Chemicals of Mount Holly, N.C. U.S.A. and Glucopan 220UP from Henkel Corporation of Ambler, Pa. in an active ratio of 3:1. The surfactant can be applied by any conventional means, such as spraying, printing, brush coating or the like. The surfactant can be applied to the entire bodyside liner 42 or can be selectively applied to particular sections of the bodyside liner, such as the medial section along the longitudinal centerline 36.

A suitable liquid permeable bodyside liner 42 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber available from CHISSO Corporation, Osaka, Japan. In this particular bicomponent fiber, the polypropylene forms the core and the polyethylene forms the sheath of the fiber. Other fiber orientations are possible, such as multi-lobe, side-by-side, end-to-end, or the like.

The absorbent assembly 44 (FIG. 3) is positioned between the outer cover 40 and the bodyside liner 42, which components can be joined together by any suitable means such as adhesives as is well known in the art. The absorbent assembly 44 can be any structure which is generally compressible, conformable, non-irritating to the child's skin, and capable of absorbing and retaining liquids and certain body wastes. The absorbent assembly 44 can be manufactured in a wide variety of sizes and shapes, and from a wide variety of liquid absorbent materials commonly used in the art. For example, the absorbent assembly 44 can suitably comprise a matrix of hydrophilic fibers, such as a web of cellulosic fluff, mixed with particles of a high-absorbency material commonly known as superabsorbent material. In a particular embodiment, the absorbent assembly 44 comprises a matrix of cellulosic fluff, such as wood pulp fluff, and superabsorbent hydrogel-forming particles. The wood pulp fluff can be exchanged with synthetic, polymeric, meltblown fibers or short cut homofil bicomponent synthetic fibers and natural fibers. The superabsorbent particles can be substantially homogeneously mixed with the hydrophilic fibers or can be nonuniformly mixed. The fluff and superabsorbent particles can also be selectively placed into desired zones of the absorbent assembly 44 to better contain and absorb body exudates. The concentration of the superabsorbent particles can also vary through the thickness of the absorbent assembly 44. Alternatively, the absorbent assembly 44 can comprise a laminate of fibrous webs and superabsorbent material or other suitable means of maintaining a superabsorbent material in a localized area.

Suitable superabsorbent materials can be selected from natural, synthetic, and modified natural polymers and materials. The superabsorbent materials can be inorganic materials, such as silica gels, or organic compounds, such as crosslinked polymers. Suitable superabsorbent materials are available from various commercial vendors, such as Dow Chemical Company located in Midland, Mich. U.S.A. and Stockhausen GmbH & Co. KG, D-47805 Krefeld, Federal Republic of Germany. Typically, a superabsorbent material is capable of absorbing at least about 15 times its weight in water, and desirably is capable of absorbing more than about 25 times its weight in water. In one embodiment, the absorbent assembly 44 is generally rectangular in shape, and comprises a blend of wood pulp fluff and superabsorbent material. One preferred type of fluff is identified with the trade designation CR1654, available from U.S. Alliance, Childersburg, Ala. U.S.A., and is a bleached, highly absorbent sulfate wood pulp containing primarily soft wood fibers and about 16 percent hardwood fibers. As a general rule, the superabsorbent material is present in the absorbent assembly 44 in an amount of from about 5 to about 90 weight percent based on total weight of the absorbent assembly. The absorbent assembly 44 suitably has a density within the range of about 0.10 to about 0.35 grams per cubic centimeter. The absorbent assembly 44 may or may not be wrapped or encompassed by a suitable tissue wrap that maintains the integrity and/or shape of absorbent assembly.

The absorbent chassis 32 can also incorporate other materials that are designed primarily to receive, temporarily store, and/or transport liquid along the mutually facing surface with absorbent assembly 44, thereby maximizing the absorbent capacity of absorbent assembly. One suitable material is referred to as a surge layer (not shown) and comprises a material having a basis weight of about 50 grams per square meter, and comprising a through-air-bonded-carded web of a homogenous blend of 60 percent 3 denier bicomponent fiber comprising a polyester core/polyethylene sheath, commercially available from BASF Corporation, and 40 percent 6 denier polyester fiber, commercially available from Hoechst Celanese.

As noted previously, the illustrated training pant 20 has a side panel 34 disposed on each side of the absorbent chassis 32. In the illustrated embodiment, the pair of transversely opposed side panels 34 are permanently bonded to the absorbent chassis 32 and permanently bonded to one another, using attachment means known to those skilled in the art such as adhesive, thermal or ultrasonic bonding.

The side panels 34 desirably comprise an elastic material capable of stretching in a direction parallel to the transverse axis 49 of the training pant 20. Suitable elastic materials, as well as one described process of incorporating elastic side panels into a training pant, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. In particular embodiments, the elastic material comprises a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; and European Patent Application No. EP 0 217 032 published on Apr. 8, 1987 in the names of Taylor et al.; all of which are incorporated herein by reference. Alternatively, the side panel material can comprise other woven or nonwoven materials, such as those described above as being suitable for the outer cover 40 or bodyside liner 42.

The various components of the training pant 20 are integrally assembled together employing various types of suitable attachment means, such as adhesive, sonic and thermal bonds or combinations thereof. The graphics can be formed by any suitable is method, including but not limited to printing, such as flexographic, rotogravure, or ink-jet; assembling a multiple layer composite with at least one of the layers containing the graphics; or the like.

### EXAMPLES

The following examples serve to illustrate possible approaches pertaining to the present invention. The particular amounts, proportions, compositions and parameters are meant to be exemplary, and are not intended to specifically limit the scope of the invention.

Example 1 of the present invention is a spunbond web consisting of 100 percent polypropylene resin, which is suitable for use as the outer layer of an outer cover. The web had a basis weight of 18.7 grams per square meter and a wire weave bond pattern. Five specimens were tested and found to have opacity values measured in percent light blocked of 14.8, 12.4, 15.8, 11.2 and 15.2, giving an average of 13.88 percent and a standard deviation of 1.98. The resulting Light Transmittance Value for Example 1 is 86.12 percent.

Example 2 is a comparative example measuring the Light Transmittance Value of a spunbond web consisting of 99 weight percent polypropylene resin and 1 weight percent titanium dioxide. Similar web materials have been used by Kimberly-Clark Corporation for the outer layer of the outer cover of HUGGIES® PULL-UPS® training pants in the United States during 1999. Five specimens were tested and found to have opacity values measured in percent light blocked of 29.1, 27.1, 26.3, 25.6 and 24.6, giving an average of 26.54 percent and a standard deviation of 1.7. The resulting Light Transmittance Value for Example 2 is 73.46 percent.

Example 3 is a comparative example measuring the Light Transmittance Value of the outer nonwoven layer of the outer cover of a training pant sold by Paragon in the United States during 1999. The outer nonwoven layer was hand separated from a film inner layer of the training pant outer cover prior to measurement. Five specimens were tested and found to have opacity values measured in percent light blocked of 26.2, 24.2, 26.2, 25.8 and 27.5, giving an average of 25.98 percent and a standard deviation of 1.18. The resulting Light Transmittance Value for Example 3 is 74.02 percent.

The following procedure can be used to determine the Light Transmittance Value of a material. The person using this procedure should be trained on proper use of the equipment.

1. Apparatus and Materials

1.1 BYK-Gardner TCS® Color Sphere Spectrophotometer, which is a high-performance, rapid measuring spectrophotometer using a d/8° geometry (diffuse illumination and 8° viewing), with instrument operation manual and software; available from BYK-Gardner, Inc., Columbia, Md. U.S.A. The equipment should be in good condition and be properly calibrated.

1.2 Menu driven quality control software program; available from BYK-Gardner, Inc.

1.3 Small area lens and aperture; available from BYK-Gardner, Inc.

1.4 Quartz halogen lamp with infrared filter; available from BYK-Gardner, Inc.

1.5 Black cavity; available from BYK-Gardner, Inc.

1.6 White Japanese opal standard, with calibration data; available from BYK-Gardner, Inc.

1.7 Personal computer, meeting minimum requirements: 386 processor, 2 MB RAM, 10 MB or more available on hard drive, DOS 5.0 or higher, Microsoft WINDOWS 3.0, Microsoft WINDOWS supported printer, Microsoft Mouse, math coprocessor, and EXCEL for WINDOWS.

1.8 Power surge protector.

1.9 Cutting device, such as a scissors or paper cutter capable of cutting specimens to the requisite dimensions as specified below.

2. Conditioning:

Testing should be conducted in a standard laboratory atmosphere of 23°±2° C. (73.4°±3.6° F.) and 50±5% relative humidity.

3. Test Specimen:

For each material to be tested, cut five specimens of approximately 51 by 51 millimeters (2 by 2 inches).

4. Preparation of Apparatus and Materials:

Consult the Color Sphere Spectrophotometer operation manual for software installation, system configuration, and instrument interface to the computer. Always keep lenses, heat filters, and mirrors in the optical unit free of dust and dirt. Keep foreign material and liquids from getting into the sphere.

4.1 Plug the computer, printer, and the spectrophotometer into the same surge protector.

4.2 Turn the surge protector power on so the computer and the spectrophotometer are powered up at the same time, a minimum of 30 minutes prior to testing.

4.3 Open the sample clamp and screw the large area aperture onto the reflectance port. Care should be exercised when handling any aperture or cover, as each one is coated with barium sulfite that can chip or discolor easily. Ensure the small aperture fixture is not in the transmission compartment and the lens for large area aperture is in the correct position and secure.

4.4 At the WINDOWS Program Manager screen, find the COLOR-INSIGHTS box. Double click on the A QC MANAGER icon.

4.5 Click on EDIT.

4.6 Click on SETTINGS. The following configuration should be used:

4.6.1 Color Scale

4.6.2 Display: XYZ

4.6.3 Illuminant: C

4.6.4 Observer: 2°

4.7 Click on OK.

4.8 Click on FILE.

4.9 Click on CALIBRATE. At the TCS® Calibrate Screen, verify that the set-up on the screen is consistent with the actual instrument set-up. The default should be as follows: Reflectance, Large Port Size, and Specular Included.

4.10 Click on OK or press ENTER.

4.11 Hold the black cavity against the reflectance port.

4.12 Click on OK or press ENTER.

4.13 Hold the white Japanese opal standard tile against the reflectance port. Place the tile so that the 2 pegs are resting on top of the reflectance port cover plate.

4.14 Click on OK or press ENTER.

4.15 Click on DISPLAY.

4.16 Click on DATA.

4.17 Double click on STANDARD or press ENTER.

4.18 Compare the nanometer results to the number supplied by BYK-Gardner for that tile. Nanometer numbers for 380, 540, and 720 nm must be ±0.25 nanometer. If the result is greater than ±0.25 nm, repeat the calibration. If after a second calibration, the results are still not within ±0.25 nm, contact BYK-Gardner.

4.19 Remove the white Japanese opal standard tile from the reflectance port.

4.20 Click on DISPLAY.

4.21 Click on SCALE.

4.22 Click on DISPLAY and then on OPACITY. Note that multiple samples must be off in order to select OPACITY.

4.23 If prompted to save changes, select NO.

4.24 Check DISPLAY WHITE under standard and sample. The Opacity measurement consists of measurement of a specimen backed with a black reference and the measurement of the same specimen backed with a white reference. The Color measurement of the specimen can be displayed with either the black reference or the white reference as the backing. Select DISPLAY WHITE for the Sample and Standard columns.

4.25 Click on ON.

5. Procedure

5.1 To take a measurement (in Opacity mode) double-click on the sample or standard column or press ENTER. Note that the system will display a READY TO MEASURE prompt for Standard (Specimen) backed with the black cavity.

5.2 Center a single specimen over the reflectance port making sure there are no wrinkles and avoid large embossed areas.

5.3 Back the specimen with the black cavity.

5.4 Click on OK or press ENTER. Note that the system will display a READY TO MEASURE prompt for specimen with white reference.

5.5 Place the white reference (white side of black cavity) over the specimen on the reflectance port. Do not move the specimen test area between the black cavity reading and the white reference reading.

5.6 Click on OK or press ENTER.

5.7 Record the C2° Opacity result.

5.8 Repeat 5.1 through 5.7 for each specimen.

5.9 Turn off Opacity mode when finished by clicking on DISPLAY and then OPACITY!. Then click on OFF. Click on YES. The system will then exit the Opacity mode.

5.10 If prompted to save changes, select NO.

6. Results

6.1 Record the entire equipment set-up; e.g., large area view, specular included, observer, and illuminant.

6.2 Record the C2° Opacity result for every specimen.

6.3 Calculate the average and standard deviation for each group of five specimens.

6.4 The Light Transmittance Value for the material being tested is equal to 100 minus the average opacity for the group of specimens.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

We claim:

1. An absorbent article, comprising:
an absorbent assembly having a major surface;
an outer cover positioned against the major surface, the outer cover comprising a liquid impermeable inner layer and a fibrous outer layer which layers jointly define at least one graphic region where the inner and outer layers are intimately bonded together, the fibrous outer layer having a Light Transmittance Value in the at least one graphic region of about 80 percent or higher; and
an outer cover graphic disposed on the inner layer in a location that corresponds to the at least one graphic region.

2. The absorbent article of claim 1, wherein the fibrous outer layer has a Light Transmittance Value in the at least one graphic region from about 80 to about 95 percent.

3. The absorbent article of claim 2, wherein the fibrous outer layer has a Light Transmittance Value in the at least one graphic region from about 82 to about 95 percent.

4. The absorbent article of claim 1, wherein the fibrous outer layer has a Light Transmittance Value in the at least one graphic region of about 84 percent or higher.

5. The absorbent article of claim 4, wherein the fibrous outer layer has a Light Transmittance Value in the at least one graphic region from about 84 to about 88 percent or higher.

6. The absorbent article of claim 1, wherein the outer cover layer comprises a polymer resin and about 0.5 weight percent or less other additives.

7. The absorbent article of claim 6, wherein the outer cover layer comprises about 0.3 weight percent or less other additives.

8. The absorbent article of claim 6, wherein the outer layer comprises a spunbond web formed of polypropylene resin.

9. The absorbent article of claim 1, wherein the outer layer comprises a polymer resin and about 0.5 weight percent or less titanium dioxide.

10. The absorbent article of claim 9, wherein the outer layer comprises about 0.3 weight percent or less titanium dioxide.

11. The absorbent article of claim 1, wherein the outer cover graphic is disposed directly on a layer of the outer cover.

12. The absorbent article of claim 11, wherein the outer cover graphic is disposed directly on the inner layer.

13. The absorbent article of claim 11, wherein the outer cover graphic is disposed directly on an interior surface of the outer layer.

14. The absorbent article of claim 1, wherein the outer cover graphic is disposed directly on a layer placed adjacent the inner layer.

15. The absorbent article of claim 1, wherein the outer cover graphic is located between the inner and outer layers.

16. The absorbent article of claim 1, wherein the bonded surface area between the inner and outer layers is about 2 percent or more in the at least one graphic region.

17. The absorbent article of claim 1, wherein the outer layer has a basis weight of about 13 to about 34 grams per square meter.

18. The absorbent article of claim 1, wherein the inner layer comprises a liquid impermeable polymeric film.

19. An absorbent article, comprising:
an absorbent assembly;
a liquid permeable topsheet disposed on the absorbent assembly; and
an outer cover bonded to the topsheet and sandwiching the absorbent assembly between the topsheet and the outer cover, the outer cover comprising a liquid impermeable inner layer and a fibrous outer layer which layers jointly define at least one graphic region where the inner and outer layers are intimately bonded together, the fibrous outer layer having a Light Transmittance Value in the at least one graphic region of about 82 percent or higher; and
an active graphic disposed on the inner layer in liquid communication with the absorbent assembly and in a location that corresponds to the at least one graphic region.

20. The absorbent article of claim 19, wherein the fibrous outer layer has a Light Transmittance Value in the at least one graphic region of about 84 percent or higher.

21. The absorbent article of claim 20, wherein the fibrous outer layer has a Light Transmittance Value in the at least one graphic region from about 84 to about 88 percent.

22. The absorbent article of claim 19, wherein the outer cover layer comprises a polymer resin and about 0.5 weight percent or less other additives.

23. The absorbent article of claim 22, wherein the outer cover layer comprises about 0.3 weight percent or less other additives.

24. The absorbent article of claim 22, wherein the outer layer comprises a spunbond web formed of polypropylene resin.

25. The absorbent article of claim 19, wherein the outer cover layer comprises a polymer resin and about 0.5 weight percent or less titanium dioxide.

26. The absorbent article of claim 25, wherein the outer cover layer comprises about 0.3 weight percent or less titanium dioxide.

27. The absorbent article of claim 19, wherein the inner and outer layers are bonded together by thermal bonds.

28. The absorbent article of claim 19, wherein the inner and outer layers are bonded together by adhesive bonds.

29. The absorbent article of claim 19, wherein the active graphic comprises an appearing graphic.

30. The absorbent article of claim 19, wherein the active graphic comprises a fading graphic.

31. The absorbent article of claim 30, wherein the fading graphic comprises an ink that is soluble in aqueous solutions.

32. The absorbent article of claim 19, wherein the active graphic comprises a composition that changes color when exposed to an aqueous solution.

33. The absorbent article of claim 19, wherein the active graphic is disposed directly on the inner layer.

34. The absorbent article of claim 19, wherein the active graphic is disposed directly on a layer placed adjacent the inner layer.

35. The absorbent article of claim 19, wherein the bonded surface area between the inner and outer layers is about 2 percent or more in the at least one graphic region.

36. The absorbent article of claim 19, wherein the outer layer has a basis weight of about 17 to about 20 grams per square meter.

37. The absorbent article of claim 19, wherein the inner layer comprises a liquid impermeable polymeric film.
